(19) United States
(12) Patent Application Publication
(10) Pub. No.: US 2005/0249804 A1
(43) Pub. Date: Nov. 10, 2005

(54) Stabilized short-course chemotherapy (SCC) anti-tuberculosis drug compositions

(76) Inventor: Vinay Ramakant Sapte, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/841,057

(22) Filed: May 7, 2004

(51) Int. Cl. A61K 31/4965; A61K 31/496; A61K 31/355

(52) U.S. Cl. 424/464; 514/252.13; 514/255.06; 514/356

(57) ABSTRACT

A stabilized oral powder or granule mixture made from at least two different anti-microbial tuberculosis drugs (e.g., rifampacin, isoniazid, ethambutol, pyrazinamide), for a short-course therapy; the powder can be consumed by mixing in a glass of water or juice and assures that each of the various drugs is in fact consumed by the tuberculosis patient.

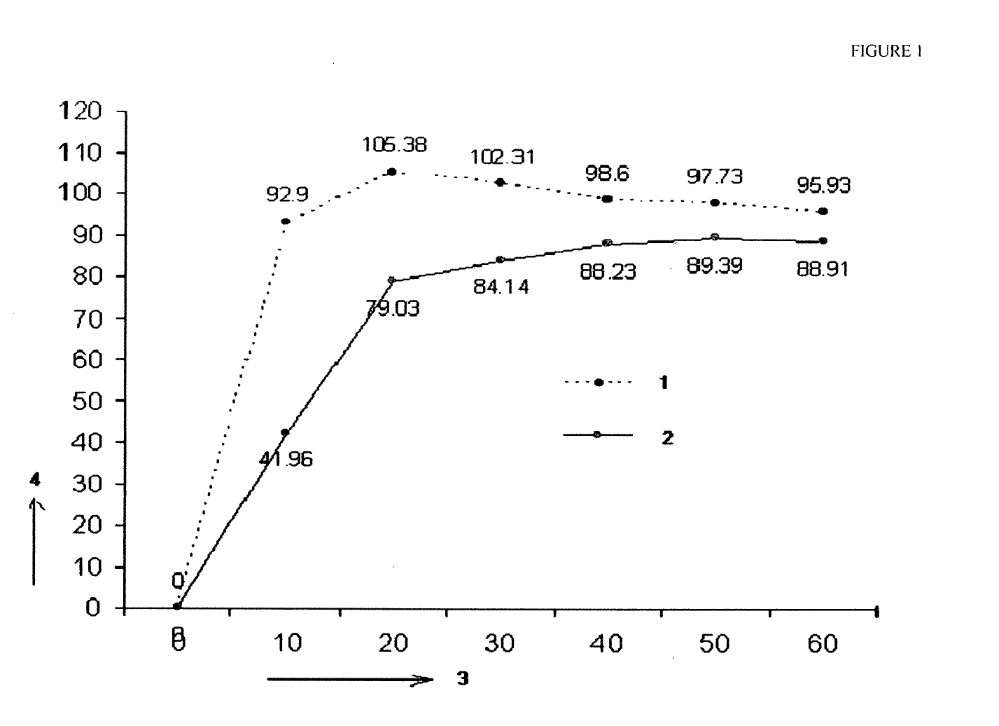

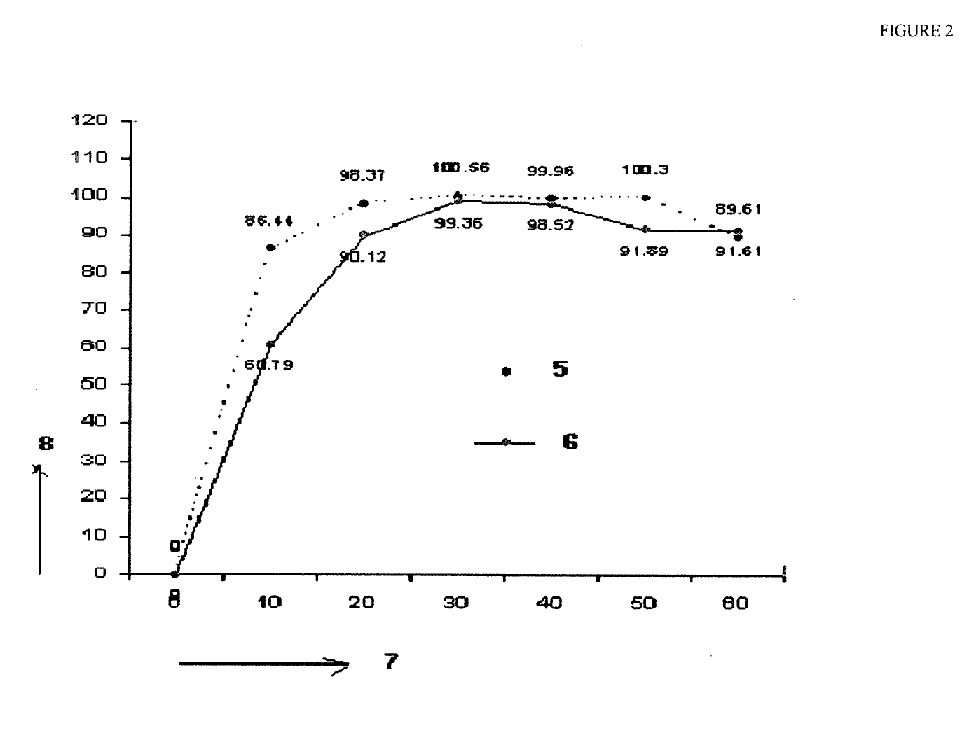

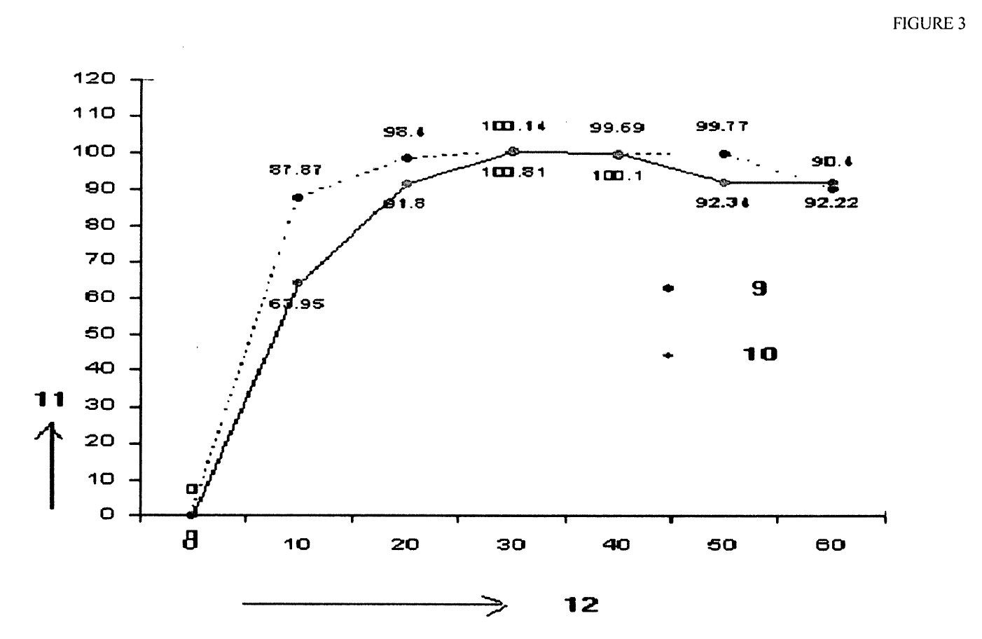

### TECHNICAL FIELD OF INVENTION

The invention relates to oral powder/granule compositions comprising up to 4 anti-TB drugs used in the short Course Chemotherapy (SCC) namely Rifampicin, Isoniazid, Ethambutol and Pyrazinamide (SCC-4), in palatable powder form, which can be consumed by mixing the powder in a glass of water or juice with meal. This invention further relates to oral/powder/granule compositions of two (SCC-2), three (SSC-3) and four (SCC-4) anti-TB drugs for short course chemotherapy (SCC).

### I. BACKGROUND OF INVENTION

Tuberculosis is one of the most common infectious diseases known to man. About 32% of the world's population is infected with TB. Every year, approximately 8 million of these infected people develop active TB and almost 2 million of these will die from the disease. In India alone, one person dies of TB every minute.

Though there are effective treatments available using the four drugs namely Rifampicin, Isoniazid, Ethambutol and Pyrazinamide, the high dose of the treatment and its long duration i.e. at least 8 weeks of Intensive phase and 12 weeks of Continuation phase results in poor compliance from TB patients. The patient is required to consume typically 6 to 8 tablets at a time on Empty stomach every day. The poor Compliance to adhere to the strict regimen has increased the incidence of Multi drug Resistant TB and relapse cases.

The failure of anti-tubercular therapy is essentially due to non-compliance or partial compliance with the recommended therapy (Tubercle and Lung Disease, 74, 32, 1993).

It has been found that partial adherence to therapy is a grave menace to community because the patient who does not take any therapy at all, transmits non-resistant tubercle bacilli to others, whereas the patient, who takes partial therapy develops multi-drug resistance and transmits drug-resistant tubercle bacilli.

Emergence of drug resistance in high burden areas of the world presents a major threat to the future success of TB control. Drug resistance in most tuberculosis patients predominantly arises as a result of multiple interruptions of treatment. When using single drug formulations, patients are more prone to interrupt their treatment on some drugs while not on others, thereby creating a risk of monotherapy and selection of drug-resistant mutants. Furthermore, out-of-stock of expiry situations in treatment facilities, which might lead to some drugs being continued in isolation while new stocks of others are being awaited, represent another potential source of monotherapy. Such problems are prevented more easily if fixed dose combinations (FDCs) are used.

In order to control re-emergence of drug resistant tuberculosis, World Health Organization (WHO) put forward a number of guidelines for effective treatment of tuberculosis, which include the following: “Directly Observed Therapy” (DOT) which requires complete supervision (Weis S. E. et. al., New Engl. J. Med., 330,1179,1994).

To improve patient compliance, minimize drug resistance and for the ease of administration, the use of fixed dose combination has been recommended by World Health Organization (WHO), Center for Disease Control (CDC), International Union Against Tuberculosis and Lung Disease (IUATLD) and, American Thoracic Society [Statement of IUATLD and WHO in ‘Tubercle and Lung Disease’ 75, 180, 1994; Moulding T. et. al., Ann. Intern. Med., 122, 951, 1995]. Tuberculosis needs the treatment with three to five different drugs simultaneously, depending upon the patient category. These anti-tuberculosis drugs can be given as single drug formulations or as fixed dose combinations (FDCs) where two or more anti-tuberculosis drugs are present in fixed proportions in the same formulation. WHO and IUATLD advocate the replacement of single drug preparations by FDC tablets as the primary treatment for tuberculosis.

In the year 1998, the World Health Organisation recommended the 4 drugs Fixed Dose Combination to increase the compliance by reducing the number of Tablets requires to be consumed by the patient. Such Tablets are now available, but due to high dosage the size of the Tablets is very big and still the number of tablets required to consume at a time is at least three. Also the process of making such tablets is tricky and may result in poor Bio-availability of the drugs.

The disadvantage of the 4 FDC tablets is that, if the patient does not take all the tablets i.e. three or four as recommended at a time, as per the body weight the dose becomes sub-optimal and there is then the risk of developing the MDR TB

MOREOVER these FDC'S are only effective if the individual components are available in tissue at the correct concentration. A number of studies have shown that, if formulation/processes are not adequately optimized, such preparations can have serious limitations and may risk the possibility of adverse treatment results and the development of drug resistance. Ensuring a reliable quality medication is one of the corner stones of tuberculosis control, the major concern in using FDCs is quality because the use of sub standard FDCs may result in treatment failure and the emergence of drug resistance.

The major quality issue with FDC tablets is assuring the bioavailability of rifampicin. It is known that when rifampicin is combined with other drugs in the same formulation, its bioavailability is negatively affected if formulation/processes are not optimized and quality of active drugs is not controlled.

In a symposium on quality control of anti-TB drugs, at annual meeting of IUATLD in Dubrovnik in 1988, Acocella (University of Pavia, Italy) presented studies on bioavailability of rifampicin in two and three-drug FDC tablets (Acocella G., Bull. Int.

Union Tuberc. Lung Dis., 64,38,1989). His work showed that the bioavailability of rifampicin when given as FDC tablets, particularly the three-drug combination, could be poor. Furthermore, an apparently satisfactory in-vitro dissolution test does not guarantee acceptable rifampicin bioavailability. The results of a series of studies have shown that while some FDC formulations had acceptable rifampicin bioavailability, others did not.

FDC tablets gives with poor rifampicin bioavailability means giving inadequate therapy, without even being aware of it. Consequently, using FDC tablets of poor rifampicin bioavailability could directly lead to poor treatment outcome and may create, and not prevent, drug resistance. Good quality FDC tablets with demonstrated bioavailability of rifampicin, is an absolute requirement for successful treatment outcomes in programmes utilizing FDC-based regimens.

Bioavailability problems with the isoniazid, pyrazinamide and ethambutol components of FDC tablets have not been encountered, presumably because of their much greater water-solubilities. It is assumed that impaired bioavailability may result from changes in rifampicin's crystalline form during the tabletting process.

Besides being poorly soluble in water, the absorption of rifampicin is adversely affected by food. Rifampicin alone, in solid state, is stable but its stability in the presence of moisture and other anti-tubercular drugs together is questionable. Rifampicin is incompatible with isoniazid in presence of water (Ved S. and Deshpande S. G., Eastern Pharmacist, 139, July 1990). Ethambutol hydrochloride, which is a highly hygroscopic material, tends to catalyze rifampicin and isoniazid interaction. Hence the development of four-drug FDCs containing rifampicin demands not only improving the solubility of rifampicin but also protecting it against oxidation and interaction with the other drugs.

The two, three and four-drug FDCs recommended by WHO and included in the WHO model list of essential drugs contain varying compositions of each drug based on the age, gender and weight of the patients they are intended for. To ensure that the process used for manufacturing the entire range of FDCs with variable active ingredient compositions is economically viable, a flexible process by means of which all the different compositions can be manufactured must be available.

Japanese Patent No. 53-133624 discloses a formula for overcoming poor elution properties of solid pharmaceutical preparations containing rifampicin. Capsules containing mixtures of rifampicin with crystalline cellulose alone or with crystalline cellulose together with polyethyleneglycol 40 monostearate, polyethyleneglycol 80 sorbitan monooleate, glycerol monostearate, hydroxypropyl cellulose or hydroxypropyl methylcellulose and magnesium stearate showed satisfactory elution properties when tested in a medium with a pH of 1.5 or 3, using the rotating basket method.

U.S. Pat. No. 4,613,496 teaches that while the compositions described in the above Japanese patent show a considerable improvement of elution properties over those of ordinary preparations, it has been found that these properties are no longer satisfactory under neutral to slightly basic conditions when the elution rates are determined with the column dissolution rate testing method which more accurately reflects the actual physiological conditions prevailing in the human body than the rotating basket method.

U.S. Pat. No. 4,613,496 discloses capsules containing a mixture of rifampicin, crystalline cellulose, sodium lauryl sulfate and magnesium stearate, which show consistently more uniform and more complete dissolution rates using the column method than those of the compositions disclosed in the above Japanese patent.

U.S. Pat. No. 5,104,875 discloses combination preparations containing rifampicin and thioacetazon and optionally isonicotinic acid hydrazide or ethambutol and its use for the treatment of mycobacterial infections.

U.S. Pat. No. 6,107,276 discloses a technique for improving the dissolution of slightly soluble drugs by employing a water-swellable, but water-insoluble cross-linked polymer, a surface-active agent and an oil mixed with the drug for improving its bioavailability.

European Patent EP 330284 B1 discloses a wet granulation process for making good quality granulate comprising of a drug present in high concentration but having limited solubility in water of less than 10 wt %, 20-100 wt % of microcrystalline cellulose or microfine cellulose or a mixture of both and 0-0.5 wt % of a wet granulation binding substance. These granulates can be processed to solid tablets having a satisfactory disintegration behavior. The text on page 4, lines 26-30, further elaborates the limitation of the invention, that the use of a wet granulation binding substance in the granulation mixture should be avoided or at least restricted to an amount of not more than 0.5 wt %, preferably to less than 0.1 wt % based on the weight of the drug. Otherwise the disintegration behavior of the tablets prepared from these granulates is adversely affected.

PCT patent application WO 98/06382 discloses a granulate consisting of water soluble active ingredient at least 75 wt %, up to and including 100 wt % of a microcrystalline cellulose, and up to and including 0.5 wt % of a wet granulation binding agent prepared at room temperature by a wet granulation technique.

The Indian Patent No. 181730 discloses a wet granulation process for manufacture of tablets containing rifampicin, isoniazid, pyrazinamide and ethambutol hydrochloride along with pharmaceutically acceptable excipients, stabilizers and non-ionic surfactants.

This four-drug FDC is claimed to exhibit stability and bioavailability, which is comparable with single drug dosage forms containing equivalent amount of the drugs.

Two processes are described for manufacture of four-drug FDCs. In one process, rifampicin and ethambutol hydrochloride are to be wet granulated with excipients and isoniazid and pyrazinamide wet granulated with excipients followed by mixing and compression of granules obtained in these two steps. The other process teaches wet granulating rifampicin separately with excipients and the other 3 drugs together with excipients, mixing and compression of granules obtained in these two steps. These processes are hereinafter referred to as 2-step granulation processes. The disadvantages of the processes described lie in the fact that since 2 or more ingredients are granulated together, it is not possible to use the same granules to manufacture other FDCs having different strengths of the drugs.

### OBJECTIVES OF THE INVENTION

As evident from the prior art, it becomes challenging to formulate a composition containing granules of water-soluble drugs like ethambutol hydrochloride and isoniazid as well as of drugs having poor water solubility like rifampicin and pyrazinamide and still get a composition having good disintegration time.

The invented composition in powder/granule/pellet forms packed in pouches/sachets eliminates all the problems and the process requirements of wet granulation, drying, mixing & lubricating with surfactant and compressing the tablets. The invented product avoids the need for coating of the product.

The object of present invention is to provide complex each of the active drugs with agents such as Hydroxypropyl Methyl Cellulose, Ethyl Cellulose, starches or cellulosins or Schardinger sugars, which will encapsulate the particles of the Active Drug. When mixed together after such a treatment these drugs will not react with each other. Also the process of complexation is such that it helps in the better dissolution of the drug which is not easily soluble like rifampicin. Such complexation also reduces the absorption of moisture in highly hygroscopic materials like Ethambutol Hudrochloride.

Another object of present invention is to formulate the exact quantity of dosage required in one single sachet pouch thereby avoiding less than or more than optimal dosage.

Another object of the present invention was to prepare the powder in such a way that after mixing in water it will be palatable for the patient.

The invented powder composition in a Sachet or pouch form has following advantages.
1. It provides a very simple way of delivering all the four drugs in a correct dosage form in a form in a single dose.
2. The dosage is palatable and can be consumed by simply nixing in a glass of water to be taken as a Juice.
3. The increased dissolution of all for drugs ie. More than 90% in first 15 minutes as against 45 minutes for 4 drug FDC tablets.
4. The four drugs are formulated is such a way that their bio-availability is better than Tablets.
5. The sachet provided better compliance and effective therapy of all four drugs.
6. Monotheray is prevented, therby reducing the risk of drug Resistant bacilli.
7. Priscription and administration is very simple.
8. The formulation required for various weight groups as recommended by World Health Organisation is easily possible by adjusting the dosage of each drug.
9. Sachets are available for combination of all four or three or two drugs as may be required.

### SUMMARY OF INVENTION

Oral powder/granule compositions comprising up to 4 anti-TB drugs used in the short Course Chemotherapy (SCC) namely Rifampicin, Isoniazid, Ethambutol and Pyrazinamide (SCC-4), in palatable powder form, which can be consumed by mixing the powder in a glass of water or juice with meal is disclosed. This invention further discloses oral/powder/granule compositions of two (SCC-2), three (SSC-3) and four (SCC-4) anti-TB drugs for short course chemotherapy (SCC).

### DETAILED DESCRIPTION

The present invention discloses Oral powder/granule compositions comprising up to 4 anti-TB drugs used in the short Course Chemotherapy (SCC) namely Rifampicin, Isoniazid, Ethambutol and Pyrazinamide (SCC-4), in palatable powder form, which can be consumed by mixing the powder in a glass of water or juice with meal. Further oral/powder/granule compositions of two (SCC-2), three (SSC-3) and four (SCC-4) anti-TB drugs for short course chemotherapy (SCC) are also disclosed.

SCC powder composition of the Anti-tubercular drugs namely Rifampicin (R), Isoniazid (H), Pyrazinamide (Z) and Ethambutol (E) Hydrochloride packed into a pouch/sachet comprises;
1. Complexation of Rifampicin with Schardinger Sugar to form a stable complex. The particular type of Schardinger Sugar used structurally consists of 7 d-glucopyranosyl unit connected by alpha—(1, 4) glycosidic linkages. The percentage of Schardinger Sugar is 5 to 50% w/w of Rifampicin.
2. Complexation of Isoniazid with Schardinger Sugar to form a stable complex. The particular type of Schardinger Sugar used structurally consists of 7 d-glucopyranosyl unit connected by alpha—(1, 4) glycosidic linkages. The percentage of Schardinger Sugar is 5 to 50% w/w of Isoniazid.
3. Complexation of Ethambutol with Schardinger Sugar to form a stable complex. The particular type of Schardinger Sugar used structurally consists of 7 d-glucopyranosyl unit connected by alpha—(1, 4) glycosidic linkages. The percentage of Schardinger Sugar is 5 to 50% w/w of Ethambutol.
4. Complexation of Pyrazinamide with Schardinger Sugar to form a stable complex. The particular type of Schardinger Sugar used structurally consists of 7 d-glucopyranosyl unit connected by alpha—(1, 4) glycosidic linkages. The percentage of Schardinger Sugar is 5 to 50% w/w of Pyrazinamide.
5. Mixing of complexes obtained from step 1, 2, 3 & 4 with the excipients, flavours and sweetening agent in the required proportions to obtain the Final Blend for SCC 4 drugs Blend.
6. Mixing of complexes obtained from step 1, 2 & 3 with the excipients, flavours and sweetening agent in the required proportions to obtain the Final Blend for SCC 3 (RHE) drugs Blend.
7. Mixing of complexes obtained from step 1 & 2 with the excipients, flavours and sweetening agent in the required proportions to obtain the Final Blend for SCC 2 (RH) drugs Blend.
8. Mixing of complexes obtained from step 1, 2 & 4 with the excipients, flavours and sweetening agent in the required proportions to obtain the Final Blend for SCC 3 (RHZ) drugs Blend.
9. Mixing of complexes obtained from step 2 & 3 with the excepients, flavours and sweetening agent in the required proportions to obtain the Final Blend for SCC 2 (HE) drugs Blend.
10. Converting the resultant Blend from item 5,6,7,8 & 9 into a pouch or sachet form.
Note: The Schardinger Sugar used is also known as Betacyclodextrins.

### BRIEF DESCRIPTION OF DRAWINGS

FIG. I illustrates comparative in-vitro bioavailability study of Rifampicin in SCC-4 composition of the present invention and SCC-4 tablet of FDC. No 1 indicates in-vitro bioavailability of rifampicin in SCC-4 composition. In-vitro bioavailability of rifampicin in SCC-4 tablet of FDC is shown by No. 2. X-axis (No. 3) is time in minutes against Y axis (No. 4) is percentage of in-vitro bioavailable of rifampicin in the fig I.

FIG. II illustrates comparative in-vitro bioavailability study of Pyrazinamide in SCC-4 composition of the present invention and SCC-4 tablet of FDC. In-vitro bioavailability of Pyrazinamide is showned by No.5 and No. 6 in SCC-4 composition and SCC-4 tablet of FDC. In FIG. II X-axis (7) represents time in minute and Y-axis (8) is representing percentage of in-vitro bioavailability of pyrazinamide in SCC-4 composition and SCC-4 tablet of FDC.

FIG. III illustrates comparative in-vitro bioavailability study of Isoniazid in SCC-4 composition of the present invention and SCC-4 tablet of FDC. No. 9 and 10 indicates in-vitro bioavailability of isoniazid in SCC-4 composition of the present invention and SCC-4 tablet of FDC. X-axis (11) represents the time in minute while Y-axis (12) represents percentage of in-vitro bioavailability of isoniazid in SCC-4 composition and SCC-4 tablet of FDC.

### II. EXAMPLES

### Example 1

### SCC 4 Drugs (RHEZ) Sachet

| | WEIGHT | |
|---|---|---|
| INGREDIENTS | (mg/Sachet) | % W/W |
| Rifampicin | 450.00 | 14.85 |
| Isoniazid | 225 | 7.43 |
| Ethambutol | 825 | 27.23 |
| Pyrazinamide | 1200 | 39.60 |
| Betacyclodextrin | 135 | 4.46 |
| Flavour Orange | 75 | 2.46 |
| Aspartem | 120 | 3.97 |

### Example 2

### SCC 3 Drugs (RHE) Sachet

| | WEIGHT | |
|---|---|---|
| INGREDIENTS | (mg/Sachet) | % W/W |
| Rifampicin | 450.00 | 25.78 |
| Isoniazid | 225 | 12.90 |
| Ethambutol | 825 | 47.28 |
| Betacyclodextrin | 75 | 4.30 |
| Flavour Orange | 50 | 2.86 |
| Aspartem | 120 | 6.88 |

### Example 3

### SCC 2 Drugs (RH) Sachet

| | WEIGHT | |
|---|---|---|
| INGREDIENTS | (mg/Sachet) | % W/W |
| Rifampicin | 450.00 | 54.22 |
| Isoniazid | 225 | 27.11 |
| Betacyclodextrin | 35 | 4.22 |
| Flavour Orange | 40 | 4.82 |
| Aspartem | 80 | 9.63 |

While the present invention is described above in connection with preferred or illustrative embodiments, these embodiments are not intended to be exhaustive or limiting of the invention.

Rather, the invention is intended to cover all alternatives, modifications and equivalents included within its spirit and scope, as defined by appended claims.

It should be appreciated by those of skill in the art that the techniques disclosed in the examples discussed above represent techniques I have found to function well in the practice of my invention, and thus can be considered to constitute my currently-preferred modes for its practice. However, those of skill in the art should, in the light of the present disclosure, appreciate that many changes can be made in the specific embodiments which are disclosed, and still obtain a like or similar result, without departing from the spirit and scope of the invention. For example, one might readily vary the specific sugar used; as of this writing, I prefer β-cyclodextrine, but one might combine this with or substitute this with α- or γ-cyclodextrine. I thus intent the coverage of my patent to be defined not by the specific examples discussed above, but rather by the claims I append below, and their legal equivalents.

Note that in the claims I use the term “a” to include one or more than one. Thus, for example, the phrase “an antimicrobial” means one or more antimicrobials. Similarly, the phrase “a substance selected from the group consisting of: A, B and C” means one or more substances selected from that group.

The claim term “anti-microbial” encompasses, e.g., bactericidal, bacteriostatic, fungicidal, and antiviral compounds.

1. A composition of matter comprising:
(a) an anti-microbial effective amount of an anti-microbial compound, in a stable complex with
(b) Schardinger Sugar in an amount from about 5% to about 50% (w/w) of said anti-microbial compound.

2. The composition of claim 1, said anti-microbial compound selected from the group consisting of: rifampacin; isoniazid; pyrazinamide; and ethambutol.

3. The composition of claim 2, said anti-microbial compound comprising more than one compound selected from the group consisting of: rifampacin; isoniazid; pyrazinamide; and ethambutol.

4. The composition of claim 3, said anti-microbial compound comprising rifampacin and isoniazid and pyrazinamide and ethambutol.

5. The composition of claim 4 in powder form.

6. An article of manufacture comprising the composition of claim 5 contained in an envelope packet.

7. A method to treat tuberculosis comprising administering the composition of matter of claim 5 to a patient.

8. The composition of claim 2, said anti-microbial compound selected from the group consisting of: from about 60 to about 600 mg of rifampacin; from about 75 to about 700 mg of isoniazid; from about 150 to about 1,500 mg of pyrazinamide; and from about 100 to about 1,000 mg of ethambutol.

9. The composition of claim 8, said anti-microbial compound comprising more than one compound selected from the group consisting of: from about 60 to about 600 mg of rifampacin;
from about 75 to about 700 mg of isoniazid; from about 150 to about 1,500 mg of pyrazinamide;
and from about 100 to about 1,000 mg of ethambutol.

10. The composition of claim 9 in powder form.

11. The composition of claim 10, said anti-microbial compound comprising: from about 60 to about 600 mg of rifampacin; from about 75 to about 700 mg of isoniazid; from about 150 to about 1,500 mg of pyrazinamide; and from about 100 to about 1,000 mg of ethambutol.

12. An article of manufacture comprising the composition of claim 10 contained in an envelope packet.

13. A method to treat tuberculosis comprising administering the composition of matter of claim 10 to a patient.
